(19) United States
(12) Patent Application Publication
(10) Pub. No.: US 2018/0369175 A1
(43) Pub. Date: Dec. 27, 2018

(54) NUTRITIONAL SUPPLEMENT AND USE TO PREVENT NUTRITIONAL DEFICIENCIES IN PATIENTS UNDERGOING TREATMENT IN WHICH PROTON PUMP INHIBITORS ARE ADMINISTERED

(71) Applicant: BTR Group, Inc., Pittsfield, IL (US)

(72) Inventor: James Grote, Pittsfield, IL (US)

(73) Assignee: BTR Group, Inc., Pittsfield, IL (US)

(21) Appl. No.: 16/012,962

(22) Filed: Jun. 20, 2018

Related U.S. Application Data
(60) Provisional application No. 62/523,403, filed on Jun. 22, 2017.

(51) Int. Cl. A61K 31/194; A61K 33/26; A61K 31/714; A61K 31/375; A61K 9/20; A61K 45/06

(52) U.S. Cl. CPC A61K 31/194; A61K 33/26; A61K 45/06; A61K 31/375; A61K 9/20; A61K 31/714

(57) ABSTRACT

A formulation and method for treating or preventing a deficiency of certain nutrients in patients undergoing a therapy in which one or more proton pump inhibitors is administered. The formulation includes:
a) 1000 mcg of B12, as methylcobalamin;
b) 150 mg of Vitamin C;
c) 500 mg of calcium citrate;
d) 15 mg of elemental iron in a ferrous form.

The formulation may also include 10-50 mg of a proton pump inhibitor.

### CROSS-REFERENCE TO RELATED APPLICATIONS

This application claims the benefit of U.S. Provisional Patent Application Serial No. 62/523,403, filed Jun. 22, 2017, the entire contents of which are hereby incorporated herein by reference.

### FIELD OF THE INVENTION

The present invention relates generally to nutritional supplements, and more particularly to nutritional supplements useful for persons using a proton pump inhibitor.

### BACKGROUND TO THE INVENTION

Proton pump inhibitors are widely available agents that selectively and irreversibly inhibit the gastric hydrogen/potassium adenosine triphosphatase (H+/K+-exchanging ATPase), part of the ‘proton pump’ that performs the final step in the acid secretory process of the stomach. They inhibit both basal and stimulated secretion of gastric acid.

Case reports and retrospective literature reviews have suggested adverse associations between PPI utilization and certain vitamin and mineral deficiencies. However, specific therapeutic agents adapted to address those adverse associations are not available to patients needing them. For example, an extensive literature review thru July 2016 found little information on measures to mitigate chronic PPI use and essentially nothing on trials of mineral or vitamin supplementation. A more recent review on PPI safety from the pharmacy literature commented on the paucity of published data and recognized the need for a larger body of evidence. Additional recent reviews state a continued lack of comprehensive information, treatments or trials for treating difficulties arising from proton pump inhibitor therapy. In view of this understanding in the art, a need continues to exist for nutritional supplements that are specifically adapted for use by patients taking proton pump inhibitors. The present invention addresses that need.

### SUMMARY OF THE INVENTION

One aspect of the present invention provides a formulation effective for use as a human nutritional supplement. The inventive formulation consists essentially of:
a) 1000 mcg of B12, as methylcobalamin;
b) 150 mg of Vitamin C;
c) 500 mg of calcium citrate;
d) 15 mg of elemental iron in a ferrous form.

Another aspect of the present invention provides a formulation consisting essentially of:
a) 1000 mcg of B12, as methylcobalamin;
b) 150 mg of Vitamin C;
c) 500 mg of calcium citrate;
d) 15 mg of elemental iron in a ferrous form; and
e) 10-50 mg of a proton pump inhibitor.

The inventive formulations may be provided as an oral pill.

Another aspect of the present invention provides a method for treating or preventing a deficiency of certain nutrients in patients undergoing a therapy in which proton pump inhibitors are administered. The inventive method comprises:
a) determining whether a patient undergoing treatments in which a proton pump inhibitor (PPI) is administered has, or is at risk of developing, a deficiency in one or more of the nutrients in the group consisting of vitamin B12, Vitamin C, calcium, and iron;
b) administering to a patient experiencing, or determined to be at risk of developing as a consequence of that patient's PPI treatment, a deficiency of one or more of said nutrients an oral daily dosage that consists essentially of:
   i) 1000 mcg of B12, as methylcobalamin;
   ii) 150 mg of Vitamin C;
   iii) 500 mg of calcium citrate; and
   iv) 15 mg of elemental iron in a ferrous form.

A further aspect of the present invention provides another method for treating or preventing a deficiency of certain nutrients in patients undergoing a therapy in which proton pump inhibitors are administered. The inventive method comprises:
a) determining whether a patient undergoing treatments in which a proton pump inhibitor is administered has, or is at risk of developing, a deficiency in one or more of the nutrients in the group consisting of vitamin B12, Vitamin C, calcium, and iron;
b) administering to a patient experiencing, or at risk of developing as a consequence of that patient's PPI treatment, a deficiency of one or more of said nutrients an oral daily dosage that consists essentially of:
   i) 1000 mcg of B12, as methylcobalamin;
   ii) 150 mg of Vitamin C;
   iii) 500 mg of calcium citrate;
   iv) 15 mg of elemental iron in a ferrous form; and
   v) 10-50 mg of a proton pump inhibitor.

### DESCRIPTION OF THE PREFERRED EMBODIMENTS

For the purposes of promoting an understanding of the principles of the invention, reference will now be made to certain embodiments and specific language will be used to describe the same. It will nevertheless be understood that no limitation of the scope of the invention is thereby intended, with such alterations and modifications to the illustrated device being contemplated as would normally occur to one skilled in the art to which the invention relates.

The present invention relates specifically to nutritional supplements adapted to be used by patients taking a proton pump inhibitor. For purposes of this description, the term proton pump inhibitor (PPI) will refer to the benzimidazole derivatives omeprazole, lansoprazole, pantoprazole and rabeprazole, as well as tenatoprazole, which has an imidazopyridine ring in place of the benzimidazole moiety, in both their racemic and isomeric forms.

One aspect of the present invention provides nutritional supplements for use by patients undergoing PPI therapy. The nutritional supplements consist of, or consist essentially of, specific amounts of B12, vitamin C, calcium, and iron. Other embodiments of the present invention provide nutritional supplements consisting of, or consisting essentially of, specific amounts of B12, vitamin C, calcium, and iron, in combination with a PPI.

In one aspect of the present invention there is provided a nutritional supplement comprising, or consisting essentially of:
B12, as methylcobalamin, 1000 mcg;
Vitamin C, 150 mg;
Calcium, as calcium citrate, 500 mg;
Iron, as a ferrous form containing 15 mg of elemental iron.

In a further aspect of the present invention there is provided a nutritional supplement comprising, or consisting of, or consisting essentially of, of:
B12, as methylcobalamin, 1000 mcg;
Vitamin C, 150 mg;
Calcium, as calcium citrate, 500 mg;
Iron, as a ferrous form containing 15 mg of elemental iron; and
10-50 mg of a proton pump inhibitor.

The inventive nutritional supplements are formulated for use by humans. In the most preferred embodiments the supplements are formulated for daily oral dosing. Accordingly, enteral formulations, such as oral tablets, capsules, or liquids, are generally preferred. These formulations may be provided as a sustained release formulation.

Alternatively the nutritional supplements may be provided as parenteral formulations, such as injectable liquids. In general, any form that is easy to use may be provided.

The preferred formulations may include inert ingredients such as diluents and/or carriers

In a further embodiment there is provided a formulation effective for use as a human nutritional supplement comprising, or consisting of, or consisting essentially of, of:
a) 100-5000 mcg of B12, as methylcobalamin;
b) 50-1000 mg of Vitamin C;
c) 100-2500 mg of calcium citrate;
d) 5-50 mg of elemental iron in a ferrous form.

In a further embodiment there is provided a formulation effective for use as a human nutritional supplement comprising, or consisting of, or consisting essentially of, of:
a) 250-2500 mcg of B12, as methylcobalamin;
b) 50-500 mg of Vitamin C;
c) 200-1500 mg of calcium citrate;
d) 10-50 mg of elemental iron in a ferrous form.

In a further embodiment there is provided a formulation effective for use as a human nutritional supplement comprising, or consisting of, or consisting essentially of, of:
a) 500-1500 mcg of B12, as methylcobalamin;
b) 100-250 mg of Vitamin C;
c) 250-1000 mg of calcium citrate;
d) 10-25 mg of elemental iron in a ferrous form.

In a further embodiment there is provided a formulation effective for use as a human nutritional supplement comprising, or consisting of, or consisting essentially of, of:
a) 100-5000 mcg of B12, as methylcobalamin;
b) 50-1000 mg of Vitamin C;
c) 100-2500 mg of calcium citrate;
d) 5-50 mg of elemental iron in a ferrous form; and
e) 10-50 mg of a proton pump inhibitor.

In a further embodiment there is provided a formulation effective for use as a human nutritional supplement comprising, or consisting of, or consisting essentially of, of:
a) 250-2500 mcg of B12, as methylcobalamin;
b) 50-500 mg of Vitamin C;
c) 200-1500 mg of calcium citrate;
d) 10-50 mg of elemental iron in a ferrous form; and
e) 10-50 mg of a proton pump inhibitor.

In a further embodiment there is provided a formulation effective for use as a human nutritional supplement comprising, or consisting of, or consisting essentially of, of:
a) 500-1500 mcg of B12, as methylcobalamin;
b) 100-250 mg of Vitamin C;
c) 250-1000 mg of calcium citrate;
d) 10-25 mg of elemental iron in a ferrous form; and
e) 10-50 mg of a proton pump inhibitor.

Another aspect of the present invention provides a method for treating or preventing a deficiency of certain nutrients in patients undergoing a therapy in which one or more proton pump inhibitors are administered. The inventive method comprises:
a) determining whether a patient undergoing treatments in which a proton pump inhibitor is administered has, or is at risk of developing, a deficiency in one or more of the nutrients in the group consisting of vitamin B12, Vitamin C, calcium, and iron;
b) administering to a patient experiencing, or at risk of developing as a consequence of that patient's PPI treatment, a deficiency of one or more of said nutrients an oral daily dosage that comprises or consists essentially of:
   i) 1000 mcg of B12, as methylcobalamin;
   ii) 150 mg of Vitamin C;
   iii) 500 mg of calcium citrate;
   iv) 15 mg of elemental iron in a ferrous form.

Another aspect of the present invention provides a method for treating or preventing a deficiency of certain nutrients in patients undergoing a therapy in which one or more proton pump inhibitors are administered. The inventive method comprises:
a) determining whether a patient undergoing treatments in which a proton pump inhibitor is administered has, or is at risk of developing, a deficiency in one or more of the nutrients in the group consisting of vitamin B12, Vitamin C, calcium, and iron;
b) administering to a patient experiencing, or at risk of developing as a consequence of that patient's PPI treatment, a deficiency of one or more of said nutrients an oral daily dosage that comprises or consists essentially of:
   i) 100-5000 mcg of B12, as methylcobalamin;
   ii) 50-1000 mg of Vitamin C;
   iii) 100-2500 mg of calcium citrate;
   iv) 5-50 mg of elemental iron in a ferrous form.

Another aspect of the present invention provides a method for treating or preventing a deficiency of certain nutrients in patients undergoing a therapy in which one or more proton pump inhibitors are administered. The inventive method comprises:
a) determining whether a patient undergoing treatments in which a proton pump inhibitor is administered has, or is at risk of developing, a deficiency in one or more of the nutrients in the group consisting of vitamin B12, Vitamin C, calcium, and iron;
b) administering to a patient experiencing, or at risk of developing as a consequence of that patient's PPI treatment, a deficiency of one or more of said nutrients an oral daily dosage that comprises or consists essentially of:
   i) 250-2500 mcg of B12, as methylcobalamin;
   ii) 50-500 mg of Vitamin C;
   iii) 200-1500 mg of calcium citrate;
   iv) 10-50 mg of elemental iron in a ferrous form.

Another aspect of the present invention provides a method for treating or preventing a deficiency of certain nutrients in patients undergoing a therapy in which one or more proton pump inhibitors are administered. The inventive method comprises:
a) determining whether a patient undergoing treatments in which a proton pump inhibitor is administered has, or is at risk of developing, a deficiency in one or more of the nutrients in the group consisting of vitamin B12, Vitamin C, calcium, and iron;
b) administering to a patient experiencing, or at risk of developing as a consequence of that patient's PPI treatment, a deficiency of one or more of said nutrients an oral daily dosage that comprises or consists essentially of:
   i) 500-1500 mcg of B12, as methylcobalamin;
   ii) 100-250 mg of Vitamin C;
   iii) 250-1000 mg of calcium citrate;
   iv) 10-25 mg of elemental iron in a ferrous form.

Another aspect of the present invention provides a method for treating or preventing a deficiency of certain nutrients in patients undergoing a therapy in which one or more proton pump inhibitors are administered. The inventive method comprises:
a) determining whether a patient undergoing treatments in which a proton pump inhibitor is administered has, or is at risk of developing, a deficiency in one or more of the nutrients in the group consisting of vitamin B12, Vitamin C, calcium, and iron;
b) administering to a patient experiencing, or at risk of developing as a consequence of that patient's PPI treatment, a deficiency of one or more of said nutrients an oral daily dosage that comprises or consists essentially of:
   i) 1000 mcg of B12, as methylcobalamin;
   ii) 150 mg of Vitamin C;
   iii) 500 mg of calcium citrate;
   iv) 15 mg of elemental iron in a ferrous form; and
   v) 10-50 mg of a proton pump inhibitor.

Another aspect of the present invention provides a method for treating or preventing a deficiency of certain nutrients in patients undergoing a therapy in which one or more proton pump inhibitors are administered. The inventive method comprises:
a) determining whether a patient undergoing treatments in which a proton pump inhibitor is administered has, or is at risk of developing, a deficiency in one or more of the nutrients in the group consisting of vitamin B12, Vitamin C, calcium, and iron;
b) administering to a patient experiencing, or at risk of developing as a consequence of that patient's PPI treatment, a deficiency of one or more of said nutrients an oral daily dosage that comprises or consists essentially of:
   i) 100-5000 mcg of B12, as methylcobalamin;
   ii) 50-1000 mg of Vitamin C;
   iii) 100-2500 mg of calcium citrate;
   iv) 5-50 mg of elemental iron in a ferrous form; and
   v) 10-50 mg of a proton pump inhibitor.

Another aspect of the present invention provides a method for treating or preventing a deficiency of certain nutrients in patients undergoing a therapy in which one or more proton pump inhibitors is administered. The inventive method comprises:
a) determining whether a patient undergoing treatments in which a proton pump inhibitor is administered has, or is at risk of developing, a deficiency in one or more of the nutrients in the group consisting of vitamin B12, Vitamin C, calcium, and iron;
b) administering to a patient experiencing, or at risk of developing as a consequence of that patient's PPI treatment, a deficiency of one or more of said nutrients an oral daily dosage that comprises or consists essentially of:
   i) 250-2500 mcg of B12, as methylcobalamin;
   ii) 50-500 mg of Vitamin C;
   iii) 200-1500 mg of calcium citrate;
   iv) 10-50 mg of elemental iron in a ferrous form; and
   vv) 10-50 mg of a proton pump inhibitor.

Another aspect of the present invention provides a method for treating or preventing a deficiency of certain nutrients in patients undergoing a therapy in which one or more proton pump inhibitors are administered. The inventive method comprises:
a) determining whether a patient undergoing treatments in which a proton pump inhibitor is administered has, or is at risk of developing, a deficiency in one or more of the nutrients in the group consisting of vitamin B12, Vitamin C, calcium, and iron;
b) administering to a patient experiencing, or at risk of developing as a consequence of that patient's PPI treatment, a deficiency of one or more of said nutrients an oral daily dosage that comprises or consists essentially of:
   i) 500-1500 mcg of B12, as methylcobalamin;
   ii) 100-250 mg of Vitamin C;
   iii) 250-1000 mg of calcium citrate;
   iv) 10-25 mg of elemental iron in a ferrous form; and
   v) 10-50 mg of a proton pump inhibitor.

The inventive methods are intended for use by humans. Accordingly, the inventive methods use formulations that are formulated for daily oral dosing. Accordingly, enteral formulations, such as oral tablets, capsules, or liquids, are generally preferred in the formulations used in the inventive methods, and these formulations may be provided as a sustained release formulation.

Alternatively the formulations used in the inventive methods may be provided as parenteral formulations, such as injectable liquids. In general, any form that is easy to use may be provided.

The preferred formulations may include inert ingredients such as diluents and/or carriers.

While the invention has been illustrated and described in detail in the foregoing description, the same is to be considered as illustrative and not restrictive in character, it being understood that only certain preferred embodiments have been shown and described, and that all changes and modifications that come within the spirit of the invention are desired to be protected.

Additionally, it is to be appreciated that the present invention may comprise or consist of or consist essentially of any or all of the described elements. Further, any or all of the features, elements, and/or embodiments disclosed herein may be combined with any or all of the other features, elements, and/or embodiments disclosed herein to provide an invention that comprises, consists of, or consists essentially of, such features, elements, and/or embodiments.

The grammatical device “and/or” (such as in “A and/or B”) is used in this disclosure to mean A alone, or B alone, or both A and B.

1. A formulation effective for use as a human nutritional supplement, consisting essentially of:
a) 1000 mcg of B12, as methylcobalamin;
b) 150 mg of Vitamin C;
c) 500 mg of calcium citrate;
d) 15 mg of elemental iron in a ferrous form.

2. A formulation according to claim 1 wherein said formulation is provided as an oral pill.

3. A formulation according to claim 1 and further including a diluent.

4. A formulation according to claim 1 and further including a carrier.

5. A formulation effective for use as a human nutritional supplement, consisting essentially of:
a) 100-5000 mcg of B12, as methylcobalamin;
b) 50-1000 mg of Vitamin C;
c) 100-2500 mg of calcium citrate;
d) 5-50 mg of elemental iron in a ferrous form.

6. A formulation according to claim 5 wherein said supplement consists essentially of:
a) 250-2500 mcg of B12, as methylcobalamin;
b) 50-500 mg of Vitamin C;
c) 200-1500 mg of calcium citrate;
d) 10-50 mg of elemental iron in a ferrous form.

7. A formulation according to claim 6 wherein said supplement consists essentially of:
a) 500-1500 mcg of B12, as methylcobalamin;
b) 100-250 mg of Vitamin C;
c) 250-1000 mg of calcium citrate;
d) 10-25 mg of elemental iron in a ferrous form.

8. A formulation effective for use as a human nutritional supplement, consisting essentially of:
a) 100-5000 mcg of B12, as methylcobalamin;
b) 50-1000 mg of Vitamin C;
c) 100-2500 mg of calcium citrate;
d) 5-50 mg of elemental iron in a ferrous form, and
e) 10-50 mg of a proton pump inhibitor.

9. A formulation according to claim 8 wherein said supplement consists essentially of:
a) 250-2500 mcg of B12, as methylcobalamin;
b) 50-500 mg of Vitamin C;
c) 200-1500 mg of calcium citrate;
d) 10-50 mg of elemental iron in a ferrous form, and
e) 10-50 mg of a proton pump inhibitor.

10. A formulation according to claim 9 wherein said supplement consists essentially of:
a) 500-1500 mcg of B12, as methylcobalamin;
b) 100-250 mg of Vitamin C;
c) 250-1000 mg of calcium citrate;
d) 10-25 mg of elemental iron in a ferrous form, and
e) 10-50 mg of a proton pump inhibitor.

11. A formulation according to claim 8 wherein said supplement consists essentially of:
a) 1000 mcg of B12, as methylcobalamin;
b) 150 mg of Vitamin C;
c) 500 mg of calcium citrate;
d) 15 mg of elemental iron in a ferrous form, and
e) 10-50 mg of a proton pump inhibitor.

12. A method for treating or preventing a deficiency in certain nutrients in patients undergoing a therapy in which one or more proton pump inhibitors is administered, said method comprising the steps of:
a) determining whether a patient undergoing treatments in which a proton pump inhibitor is administered has, or is at risk of developing, a deficiency in one or more of the nutrients in the group consisting of vitamin B12, Vitamin C, calcium, and iron;
b) administering to a patient experiencing, or at risk of developing as a consequence of that patient's PPI treatment, a deficiency of one or more of said nutrients, an oral daily dosage that consists essentially of:
a) 100-5000 mcg of B12, as methylcobalamin;
b) 50-1000 mg of Vitamin C;
c) 100-2500 mg of calcium citrate;
d) 5-50 mg of elemental iron in a ferrous form.

13. A method according to claim 12 wherein said oral daily dosage consists essentially of:
a) 250-2500 mcg of B12, as methylcobalamin;
b) 50-500 mg of Vitamin C;
c) 200-1500 mg of calcium citrate;
d) 10-50 mg of elemental iron in a ferrous form.

14. A method according to claim 13 wherein said oral daily dosage consists essentially of:
a) 500-1500 mcg of B12, as methylcobalamin;
b) 100-250 mg of Vitamin C;
c) 250-1000 mg of calcium citrate;
d) 10-25 mg of elemental iron in a ferrous form.

15. A method according to claim 14 wherein said oral daily dosage consists essentially of:
i) 1000 mcg of B12, as methylcobalamin;
ii) 150 mg of Vitamin C;
iii) 500 mg of calcium citrate; and
iv) 15 mg of elemental iron in a ferrous form

16. A method according to claim 12 wherein said administering step comprises administering to a patient experiencing, or at risk of developing as a consequence of that patient's PPI treatment, a deficiency of one or more of said nutrients, an oral daily dosage that consists essentially of:
i) 100-5000 mcg of B12, as methylcobalamin;
ii) 50-1000 mg of Vitamin C;
iii) 100-2500 mg of calcium citrate;
iv) 5-50 mg of elemental iron in a ferrous form; and
v) 10-50 mg of a proton pump inhibitor.

17. A method according to claim 16 wherein said oral daily dosage consists essentially of:
i) 250-2500 mcg of B12, as methylcobalamin;
ii) 50-500 mg of Vitamin C;
iii) 200-1500 mg of calcium citrate;
iv) 10-50 mg of elemental iron in a ferrous form; and
v) 10-50 mg of a proton pump inhibitor.

18. A method according to claim 17 wherein said oral daily dosage consists essentially of:
i) 500-1500 mcg of B12, as methylcobalamin;
ii) 100-250 mg of Vitamin C;
iii) 250-1000 mg of calcium citrate;
iv) 10-25 mg of elemental iron in a ferrous form; and
v) 10-50 mg of a proton pump inhibitor.

19. A method according to claim 18 wherein said oral daily dosage consists essentially of:
i) 1000 mcg of B12, as methylcobalamin;
ii) 150 mg of Vitamin C;
iii) 500 mg of calcium citrate; and
iv) 15 mg of elemental iron in a ferrous form; and
v) 10-50 mg of a proton pump inhibitor.
